(19) United States
(12) Patent Application Publication
(10) Pub. No.: US 2012/0171490 A1
(43) Pub. Date: Jul. 5, 2012

(54) NOVEL POLYMORPHIC FORM OF TERIFLUNOMIDE SALTS

(75) Inventors: Ravi Ponnaiah, Vadodara (IN); Samir Patel, Vadodara (IN); Snehal Dhol, Vadodara (IN); Vishal Ray, Vadodara (IN)

(73) Assignee: ALEMBIC PHARMACEUTICALS LIMITED, Vadodara (IN)

(21) Appl. No.: 13/382,848

(22) PCT Filed: Jun. 22, 2010
(86) PCT No.: PCT/IB10/52821

§ 371 Date: Mar. 16, 2012

(30) Foreign Application Priority Data
Jul. 9, 2009 (IN) 1622/MUM/2009

(51) Int. Cl. B32B 5/16; C07C 255/44

(52) U.S. Cl. 428/402; 558/392

(57) ABSTRACT

The present invention provides a new polymorph Form I of Teriflunomide sodium and a process for preparation thereof. The present invention provides an amorphous form of Teriflunomide sodium and a process for preparation thereof. The present invention provides a new polymorph Form I of Teriflunomide potassium and a process for preparation thereof. The present invention provides an amorphous form of Teriflunomide potassium and a process for preparation thereof. The present invention also provides particle size of Teriflunomide and its salts.

### FIELD OF THE INVENTION

The present invention relates to novel polymorphic form of Teriflunomide salts and preparation thereof. Specifically present invention relates to polymorphic form of Teriflunomide alkali salts especially sodium salt and potassium salt.

### BACKGROUND OF THE INVENTION

The chemical name of Teriflunomide is 2-cyano-3-hydroxy-N-[4-(trifluoromethyl)phenyl]-2-butenamide and formula is C₁₂H₉F₃N₂O₂ and molecular weight is 270.207.

Teriflunomide is used as Immunosupressant. It acts as tyrosine kinase inhibitor. It is used in treatment of rheumatoid arthritis, autoimmune disease and multiple sclerosis.

Teriflunomide was first disclosed and claimed in U.S. Pat. No. 5,679,709 but this patent does not mention any process of preparation for salt formation.

U.S. Pat. No. 5,494,911, U.S. Pat. No. 5,990,141 disclose various processes for preparing Teriflunomide. These patents do not disclose process for preparation Teriflunomide salts or mention any its polymorphic form.

Polymorphism, the occurrence of different crystal forms, is a property of some molecules and molecular complexes. A single molecule, may give rise to a variety of crystalline forms having distinct crystal structures and physical properties like melting point, X-ray diffraction pattern, infrared absorption fingerprint, and solid state NMR spectrum, thermogravimetric analysis (“TGA”), and differential scanning calorimetry (“DSC”) which have been used to distinguish polymorphic forms.

The difference in the physical properties of different crystalline forms results from the orientation and intermolecular interactions of adjacent molecules or complexes in the bulk solid. Accordingly, polymorphs are distinct solids sharing the same molecular formula yet having distinct advantageous physical properties compared to other crystalline forms of the same compound or complex.

One of the most important physical properties of pharmaceutical compounds is their solubility in aqueous solution, particularly their solubility in the gastric juices of a patient. For example, where absorption through the gastrointestinal tract is slow, it is often desirable for a drug that is unstable to conditions in the patient's stomach or intestine to dissolve slowly so that it does not accumulate in a deleterious environment. Different crystalline forms or polymorphs of the same pharmaceutical compounds can and reportedly do have different aqueous solubility. Pharmaceutical compounds having different particle size have different dissolution property. It enlarges the repertoire of materials that a formulation scientist has available for designing, for example, a pharmaceutical dosage form of a drug with a targeted release profile or other desired characteristic.

Pharmaceutical formulation is affected by the rate of delivery or the bioavailability of the pharmaceutically active substance is the particle size. This relationship between particle size and bioavailability is well known in the pharmaceutical industry and across a range of pharmaceutical products. In 1979, studies into the effect of crystal size on the bioavailability of Benoxaprofen were conducted (Biomed Mass Spectrom., 1979 April, 6(4), pp 173-8, Wolen R L et al; J. Pharm. Sci., 1979 July, 68(7), pp 850-2, Ridolfo A S et al)

Particle sizes of substances can be measured using various commonly available methods such as measurement using light (eg. light-scattering methods or turbidimetric methods), sedimentation methods (eg. pipette analysis using an Andreassen pipette, sedimentation scales, photosedimentometers or sedimentation in a centrifugal force), pulse methods (eg. Coulter counter) or sorting by means of gravitational or centrifugal force.

While it is possible to obtain relatively small crystals of sucralose by choosing the appropriate conditions for crystallisation it is difficult to control the crystallisation process to produce small particles of a small size distribution.

There are various known methods for the control of the particle size of substances including reduction by comminution or de-agglomeration by milling and/or sieving, or particle size increase by agglomeration through granulation, blending or a mixture thereof. These methods use commonly available equipment and/or methods for the reduction or increase of the particle sizes of material. However, these techniques do not allow for the production of a substance with a very narrow, reproducible and consistent distribution of particle size without the need to reprocess, rework or destroy those particles outside of the required distribution. Thus, these processes can be time consuming and costly if reworking of the material under the desired size is not able to be performed. In those circumstances, it is common for the fine material to be destroyed or reprocessed.

Spray-drying can also be used to achieve particles in a narrow particle size distribution. However, inconsistency of the particle size of the feedstock for this process can cause problems with the apparatus such as blockage of the spray jets

In an embodiment the particle size reduction process is a milling process. In an embodiment the particle size reduction process is selected from the group consisting of jet milling, hammer milling, compression milling and tumble milling processes, most particularly a jet milling process. A fluid energy mill or “micronizer” An air jet mill is a preferred fluid energy mill. The suspended particles are injected under pressure into a recirculating particle stream. Smaller particles are carried aloft inside the mill and swept into a vent connected to a particle size classifier such as a cyclone. The feedstock should first be milled to about 150 to 850 um which may be done using a conventional ball, roller, or hammer mill. We have found that an effective method of reducing the particle size to the required dimensions is by jet milling, which utilizes fluid energy to break the crystals into fine particles. Jet mills are suitable for grinding heat sensitive materials because they have no moving parts and the slight heat generated during the grinding is compensated by the cooling effect of the fluid as it expands at the jets through which it is introduced into the grinding chamber.

It is therefore, a need to develop novel polymorphs of Teriflunomide salts and desired particle size so that it can be useful for formulation.

Present inventors have directed their research work towards developing a process for the preparation of Teriflunomide alkali metal salts and preparation of novel polymorphic forms thereof. The present inventors have prepared Teriflunomide sodium in crystalline and amorphous form. The present inventors have prepared Teriflunomide potassium in crystalline and amorphous form. The present inventors also have obtained teriflunomide & its salt with desired particle size which can be useful for formulation.

### OBJECT OF THE INVENTION

It is therefore an object of the present invention to provide new crystalline Form I of Teriflunomide sodium.

Another object of the present invention is to provide a process for preparation of new crystalline Form I of Teriflunomide sodium.

Another object of the present invention is to provide crystalline Teriflunomide sodium having particle size distribution D₁₀ less than about 20 μm, D₅₀ less than about 40 μm, and D₉₀ less than about 100 μm

Another object of the present invention is to provide an amorphous form of Teriflunomide sodium.

Another object of the present invention is to provide a process for preparation of an amorphous form of Teriflunomide sodium.

Another object of the present invention is to provide new crystalline Form I of Teriflunomide potassium.

Another object of the present invention is to provide a process for preparation of new crystalline Form I of Teriflunomide potassium.

Another object of the present invention is to provide crystalline Teriflunomide potassium having particle size distribution D₁₀ less than about 20 μm, D₅₀ less than about 40 μm, and D₉₀ less than about 100 μm

Another object of the present invention is to provide amorphous form of Teriflunomide potassium.

Another object of the present invention is to provide a process for preparation of amorphous form of Teriflunomide potassium.

Another object of the present invention is to provide a particle size of Teriflunomide.

### SUMMARY OF THE INVENTION

According to one aspect of the present invention, there is provided a new crystalline polymorphic Form I of Teriflunomide sodium characterized by an X-ray powder diffraction (XRD) pattern having peaks expressed at 2θ at about 4.0, 6.9, 8.7, 12.0, 13.0, 13.7, 15.3, 19.7, 20.6, 27.6±0.2 degrees 2θ.

The XRD of crystalline polymorphic Form I of Teriflunomide sodium is depicted in FIG. 1.

According to second aspect of the present invention, there is provided a process for preparation of a crystalline polymorphic Form I of Teriflunomide sodium comprising steps of:

(i) providing a solution of Teriflunomide sodium by dissolving Teriflunomide sodium in water;

(ii) crystallizing the product from the said solution;

isolating crystalline Form I of Teriflunomide sodium.

According to third aspect of the present invention, there is provided crystalline Teriflunomide sodium with particle size D₁₀ less than about 20 μm, D₅₀ less than about 40 μm, and D₉₀ less than about 100 μm. The required particle size is obtained by milling the compound in micronizer.

According to forth aspect of the present invention, there is provided an amorphous form of Teriflunomide sodium. The XRD of amorphous Teriflunomide sodium is depicted in FIG. 2.

According to fifth aspect of the present invention, there is provided a process for preparation of an amorphous form of Teriflunomide sodium comprising steps of:

(i) mixing Teriflunomide with sodium hydroxide solution;

(ii) lyophilizing the solution to remove water;

isolating an amorphous form of Teriflunomide sodium.

According to sixth aspect of the present invention, there is provided a new crystalline polymorphic Form I of Teriflunomide potassium characterized by an X-ray powder diffraction (XRD) pattern having peaks expressed at 2θ at about 6.4, 6.8, 9.0, 11.4, 12.8, 13.7, 14.9, 16.3, 16.8, 17.0, 18.4, 19.7, 21.3, 22.1, 24.0, 25.3, 26.6, 27.9, 28.9, 30.7±0.2 degrees 2θ.

The XRD of crystalline polymorphic Form I of Teriflunomide potassium is depicted in FIG. 3.

According to seventh aspect of the present invention, there is provided a process for preparation of a crystalline polymorphic Form I of Teriflunomide potassium comprising steps of:

(i) providing a solution of Teriflunomide potassiun by dissolving Teriflunomide potassium in water;

(ii) crystallizing the product from the said solution;

isolating the solid to give crystalline Form I of Teriflunomide potassium.

According to eighth aspect of the present invention, there is provided crystalline Teriflunomide potassium with particle size D₁₀ less than about 20 μm, D₅₀ less than about 40 μm, and D₉₀ less than about 100 μm. The required particle size is obtained by milling the compound in micronizer.

According to ninths aspect of the present invention, there is provided an amorphous form of Teriflunomide potassium. The XRD of amorphous Teriflunomide potassium is depicted in FIG. 4.

According to tenths aspect of the present invention, there is provided a process for preparation of an amorphous form of Teriflunomide potassium comprising steps of:

(i) mixing Teriflunomide with potassium hydroxide solution;

(ii) lyophilizing the solution to remove water;

isolating amorphous form of Teriflunomide potassium.

According to eleventh aspect of the present invention, there is provided Teriflunomide with particle size D₅₀ less than about 20 μm, and D₉₀ less than about 40 μm. The required particle size is obtained by milling the compound in micronizer.

### BRIEF DESCRIPTION OF THE DRAWINGS

FIG. 1 shows the X-ray powder diffraction pattern of new polymorph Form I of Teriflunomide sodium.

FIG. 2 shows the X-ray powder diffraction pattern of amorphous form of Teriflunomide sodium.

FIG. 3 shows the X-ray powder diffraction pattern of new polymorph Form I of Teriflunomide potassium.

FIG. 4 shows the X-ray powder diffraction pattern of amorphous form of Teriflunomide potassium.

### DETAILED DESCRIPTION OF THE INVENTION

The present invention provides a polymorphic crystalline Form I of Teriflunomide sodium characterized by an X-ray powder diffraction (XRD) pattern having peaks expressed at 2θ at about 4.0, 6.9, 8.7, 12.0, 13.0, 13.7, 15.3, 19.7, 20.6, 27.6±0.2 degrees 2θ.

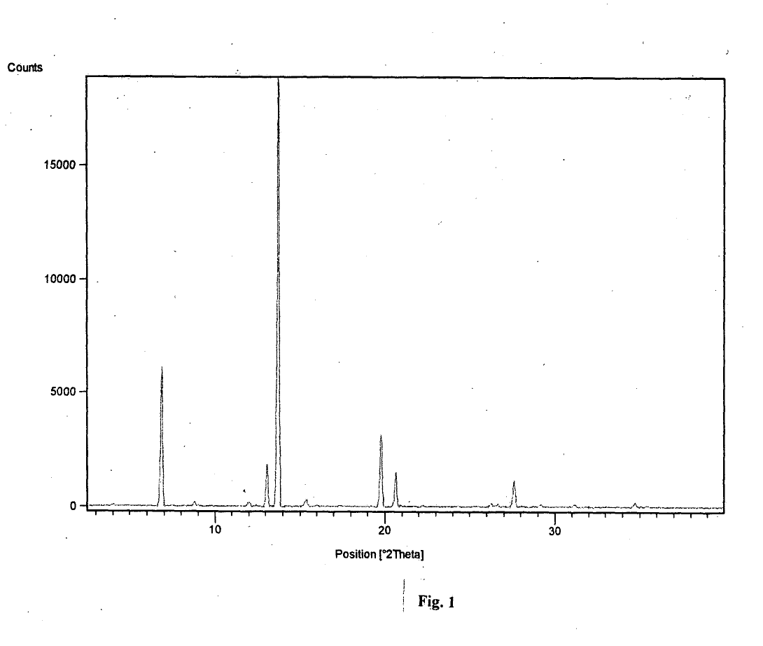

The XRD of polymorphic crystalline Form I of Teriflunomide sodium is depicted in FIG. 1.

The present invention provides a process for preparation of a crystalline Form I of Teriflunomide sodium comprising steps of:

(i) providing a solution of Teriflunomide sodium by dissolving Teriflunomide sodium in water;

(ii) crystallizing the product from the said solution;

isolating crystalline Form I of Teriflunomide sodium.

Here the term “crystallizing” means crystallizing compounds using methods known in the art. For example either reducing the volume of the solvent with respect to solute or decreasing the temperature of the solution or using both so as to crystallize the compound.

Teriflunomide sodium is dissolved in water at about 65° to 70° C. The water is taken 2 times the quantity of Teriflunomide. The solution is filtered through celite bed. The filtrate was kept overnight at room temperature for crystallization. The precipitate were filtered and dried at about 60° to 65° C. for about 12 to 14 hours to give crystalline Form I of Teriflunomide sodium.

Analysis of this solid gives XRD which is as shown in FIG. 1.

The D₁₀, D₅₀ and D₉₀ values are useful ways for indicating a particle size distribution. D₉₀ refers to the value for the particle size for which at least 90 volume percent of the particles have a size smaller than the value. Likewise D₅₀ and D₁₀ refer to the values for the particle size for which 50 volume percent, and 10 volume percent, of the particles have a size smaller than the value.

Crystalline Teriflunomide sodium as prepared according to the process of the present invention has particle side D₁₀ less than about 20 μm, D₅₀ less than about 40 μm, and D₉₀ less than about 100 μm. There is no specific lower limit for any of the D values. The required particle size is obtained by milling the compound in micronizer.

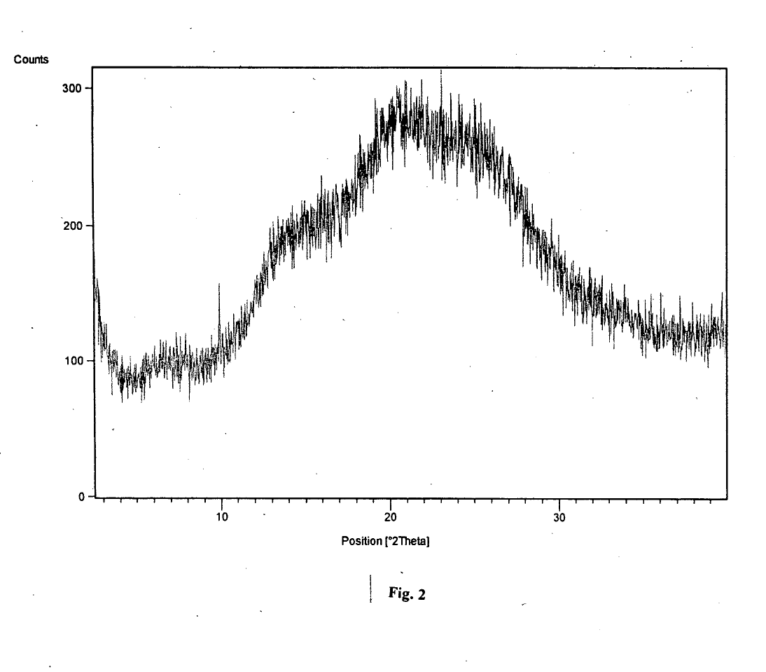

The present invention provides an amorphous form of Teriflunomide sodium. The XRD of amorphous Teriflunomide sodium is depicted in FIG. 2.

The present invention provides a process for preparation of an amorphous form of Teriflunomide sodium comprising steps of:

(i) mixing Teriflunomide with sodium hydroxide solution;

(ii) lyophilizing the solution to remove water;

isolating amorphous form of Teriflunomide sodium.

Here the term “mixing” means contacting the compound with solution which may be by means of shaking or stirring or keeping so as to the both compound and solution come in contact with each other.

Teriflunomide is added to a solution of sodium hydroxide in water. The solution is filtered through celite bed. The filtrate is concentrated using lyophilizer for about 24 hours to remove water to give amorphous form of Teriflunomide sodium.

Analysis of this solid gives XRD which is as shown in FIG. 2.

The present invention provides a polymorphic crystalline Form I of Teriflunomide potassium characterized by an X-ray powder diffraction (XRD) pattern having peaks expressed at 2θ at about 6.4, 6.8, 9.0, 11.4, 12.8, 13.7, 14.9, 16.3, 16.8, 17.0, 18.4, 19.7, 21.3, 22.1, 24.0, 25.3, 26.6, 27.9, 28.9, 30.7±0.2 degrees 2θ.

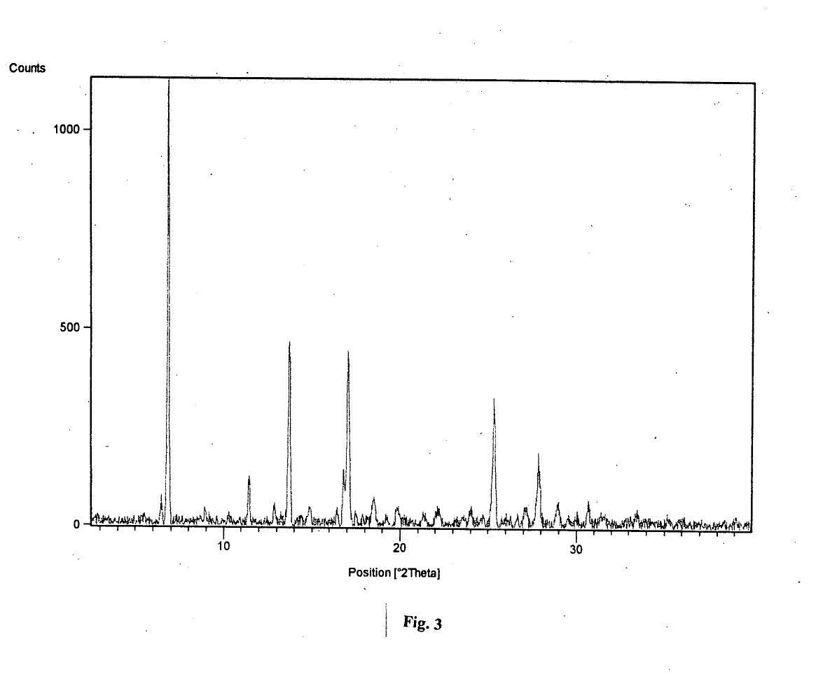

The XRD of a polymorphic crystalline Form I of Teriflunomide potassium is depicted in FIG. 3.

The present invention provides a process for preparation of a crystalline Form I of Teriflunomide potassium comprising steps of:

(i) providing a solution of Teriflunomide potassiun by dissolving Teriflunomide potassium in water;

(ii) crystallizing the product from the said solution;

isolating the solid to give crystalline Form I of Teriflunomide potassium.

Teriflunomide potassium is dissolved in water at about 65° to 70° C. The water is taken 2 times the quantity of Teriflunomide. The solution is filtered through celite bed. The filtrate was kept overnight at room temperature for crystallization. The precipitate were filtered and dried at about 60° to 65° C. for about 12 to 14 hours to give crystalline Form I of Teriflunomide potassium.

Analysis of this solid gives XRD which is as shown in FIG. 3.

Crystalline Teriflunomide potassium as prepared according to the process of the present invention has particle side D₁₀ less than about 20 μm, D₅₀ less than about 40 μm, and D₉₀ less than about 100 μm. There is no specific lower limit for any of the D values. The required particle size is obtained by milling the compound in micronizer.

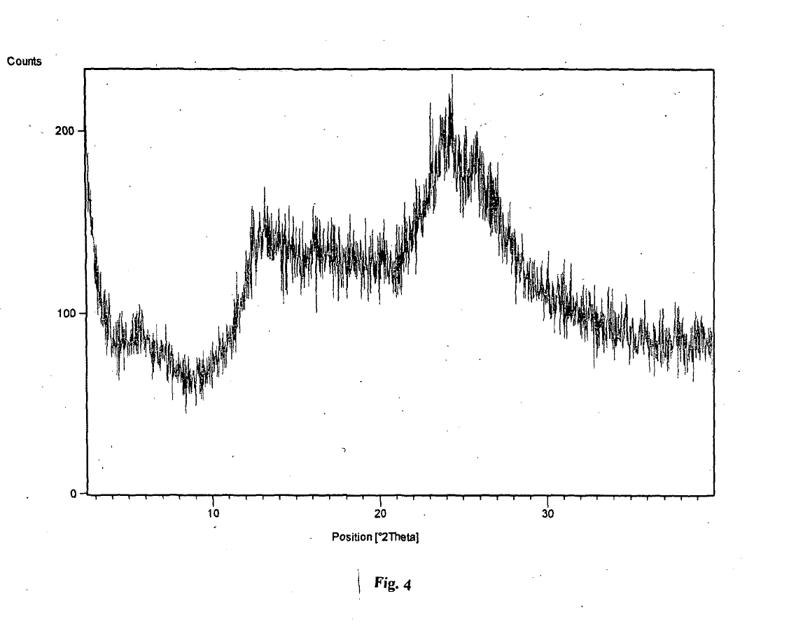

The present invention provides an amorphous form of Teriflunomide potassium. The XRD of an amorphous Teriflunomide potassium is depicted in FIG. 4.

The present invention provides a process for preparation of an amorphous form of Teriflunomide potassium comprising steps of:

(i) mixing Teriflunomide with potassium hydroxide solution;

(ii) lyophilizing the solution to remove water;

isolating amorphous form of Teriflunomide potassium.

Teriflunomide is added to a solution of potassium hydroxide in water. The solution is filtered through celite bed. The filtrate is concentrated using lyophilizer for about 24 hours to remove water to give amorphous form of Teriflunomide potassium. Analysis of this solid gives XRD which is as shown in FIG. 4.

The present invention provides Teriflunomide with particle size D₅₀ less than about 20 μm, and D₉₀ less than about 40 μm. The required particle size is obtained by milling the compound in micronizer.

The following examples illustrate the invention further. It should be understood, however, that the invention is not confined to the specific limitations set forth in the individual examples but rather to the scope of the appended claims.

### EXAMPLE 1

### Preparation of Amorphous Form of Teriflunomide Sodium

Teriflunomide (50 g) was added to a solution of sodium hydroxide (7.4 g) in water (1000 ml). The solution is filtered through celite bed. The filtrate is concentrated using lyophilizer for 24 hours to remove water to give amorphous form of Teriflunomide sodium (41.2 g)

XRD of the compound is as shown in FIG. 2

### EXAMPLE 2

### Preparation of Form I of Teriflunomide Sodium

Teriflunomide sodium (5.0 g) was dissolved in water (10.0 ml) at 60° C. The solution was filtered through celite bed. The filtrate was kept overnight at room temperature for crystallization. The precipitate were filtered and dried at 65° C. for 12 hours to give Form I of Teriflunomide sodium (2.3 g).

XRD of the compound is as shown in FIG. 1

### EXAMPLE 3

### Preparation of Amorphous Form of Teriflunomide Potassium

Teriflunomide (50 g) was added to a solution of potassium hydroxide (10.37 g) in water (1000 ml). The solution is filtered through celite bed. The filtrate is concentrated using lyophilizer for 24 hours to remove water to give amorphous form of Teriflunomide potassium (40.3 g)

XRD of the compound is as shown in FIG. 4

### EXAMPLE 4

### Preparation of Form I of Teriflunomide Potassium

Teriflunomide potassium (5.0 g) was dissolved in water (10.0 ml) at 60° C. The solution was filtered through celite bed. The filtrate was kept overnight at room temperature for crystallization. The precipitate were filtered and air dried at 65° C. for 12 hours to give Form I of Teriflunomide potassium (2.25 g).

XRD of the compound is as shown in FIG. 3

We claim:

1. A polymorphic crystalline Form I of Teriflunomide sodium characterized by an X-ray powder diffraction pattern having peaks expressed as 2θ at about 4.0, 6.9, 8.7, 12.0, 13.0, 13.7, 15.3, 19.7, 20.6, 27.6±0.2 degrees 2θ.

2. A polymorphic crystalline Form I of Teriflunomide sodium of claim 1, further characterized by an X-ray powder diffraction pattern as in FIG. 1

3. A process of preparation of a crystalline Form I of Teriflunomide sodium as claimed in claim 1 comprising steps of:
(i) providing a solution of Teriflunomide sodium by dissolving Teriflunomide sodium in water;
(ii) crystallizing the product from the said solution;
isolating crystalline Form I of Teriflunomide sodium.

4. Crystalline Teriflunomide sodium having particle size D₁₀ less than about 20 μm, D₅₀ less than about 40 μm, and D₉₀ less than about 100 μm.

5. Amorphous Teriflunomide sodium.

6. Amorphous Teriflunomide sodium of claim 5, further characterized by an X-ray powder diffraction pattern as in FIG. 2

7. A process for preparation of an amorphous form of Teriflunomide sodium comprising steps of:
(i) mixing Teriflunomide with sodium hydroxide solution;
(ii) lyophilizing the solution to remove water;
isolating amorphous form of Teriflunomide sodium.

8. A polymorphic crystalline Form I of Teriflunomide potassium characterized by an X-ray powder diffraction pattern having peaks expressed as 2θ at about 6.4, 6.8, 9.0, 11.4, 12.8, 13.7, 14.9, 16.3, 16.8, 17.0, 18.4, 19.7, 21.3, 22.1, 24.0, 25.3, 26.6, 27.9, 28.9, 30.7±0.2 degrees 2θ.

9. A polymorphic crystalline Form I of Teriflunomide potassium of claim 8, further characterized by an X-ray powder diffraction pattern as in FIG. 3

10. A process of preparation of a crystalline Form I of Teriflunomide potassium as claimed in claim 8 comprising the steps of:
(i) Providing a solution of Teriflunomide potassium by dissolving Teriflunomide potassium in water;
(ii) crystallizing the product from the said solution;
isolating crystalline Form I of Teriflunomide potassium.

11. Crystalline Teriflunomide Potassium having particle size D₁₀ less than about 20 μm, D₅₀ less than about 40 μm, and D₉₀ less than about 100 μm.

12. Amorphous Teriflunomide potassium

13. Amorphous Teriflunomide potassium of claim 12, further characterized by an X-ray powder diffraction pattern as in FIG. 4.

14. A process for preparation of an amorphous form of Teriflunomide potassium comprising steps of:
(i) mixing Teriflunomide with potassium hydroxide solution;
(ii) lyophilizing the solution to remove water;
isolating amorphous form of Teriflunomide potassium.

15. Teriflunomide having particle size D₅₀ less than about 20 μm, and D₉₀ less than about 40 μm.
